(19) United States
(12) Patent Application Publication
(10) Pub. No.: US 2025/0378942 A1
(43) Pub. Date: Dec. 11, 2025

(54) SOON-TO-EXPIRE ANALYSIS MODELS FOR MEDICAL INVENTORY MANAGEMENT

(71) Applicant: CareFusion 303, Inc., San Diego, CA (US)

(72) Inventors: Saeedeh Abbasi, San Diego, CA (US); Cynthia Yamaga, Oceanside, CA (US)

(21) Appl. No.: 18/876,093

(22) PCT Filed: Jun. 15, 2023
(86) PCT No.: PCT/US2023/025408

§ 371 Date: Dec. 17, 2024

Related U.S. Application Data
(60) Provisional application No. 63/354,915, filed on Jun. 23, 2022.

(51) Int. Cl. G16H 40/20; G06Q 10/087

(52) U.S. Cl. CPC G16H 40/20; G06Q 10/08776

(57) ABSTRACT

Methods, devices, and systems for determining soon to expire items. Historical item data are received. A soon to expire analysis model is trained using the training data to generate a trained soon to expire analysis model configured to receive item data associated with the item and to generate soon to expire prediction for one or more items associated with the item data. The bar includes a platform at a distal edge of the bar. The platform is configured to come in contact with an item deposited in the housing. A sensor is configured to generate a signal indicative of a fill level of the housing based on the platform coming in contact with the item deposited in the housing. Actions are performed to prevent the item from remaining unused past the target date.

### CROSS-REFERENCE TO RELATED APPLICATIONS

The present application claims priority to U.S. Provisional Application No. 63/354,915, filed Jun. 23, 2022, and entitled, “SOON-TO-EXPIRE ANALYSIS MODELS FOR MEDICAL INVENTORY MANAGEMENT,” the entirety of which is incorporated by reference herein.

### TECHNICAL FIELD

The subject matter described herein relates generally to data processing and more specifically to dynamic analysis models for soon-to-expire (STE) analysis and medical inventory management.

### BACKGROUND

Modern medical inventory management software applications provide a variety of solutions for monitoring the supply and usage of stored medications. These types of software applications, which are sometimes integrated with utilization management software applications and exist within a comprehensive pharmacy management software suite, may be deployed at a variety of medical settings, such as pharmacies, clinical trial labs, and healthcare facilities, to track the stocking, distribution, consumption, and disposal of various pharmaceuticals, equipment, and other supplies. In many cases, medical inventory management software applications operate to reduce operational costs and waste. For instance, many medical inventory management software applications are configured to track the shipment, delivery, storage, prescription, dispensing, administration, and wasting of individual medications while generating one or more corresponding electronic records (e.g., cost, lot number, expiration date, patient name).

### SUMMARY

Systems, methods, computer program products, and apparatuses are provided for medical inventory management with soon-to-expire (STE) analysis. In some example embodiments, an inventory controller may apply a soon-to-expire (STE) analysis model to identify one or more items that are likely to remain unused at their current stocking locations past their expiration dates. Accordingly, the inventory controller may perform one or more corrective actions to prevent the one or more items from remaining unused past their expiration dates. For example, the one or more items (or certain quantities of the one or more items) may be reallocated to a different location where they are likely to be consumed prior to their expiration dates. Alternatively and/or additionally, the inventory controller may prioritize the dispensing of the one or more items from a first location where the one or more items are more likely to remain unused past their expiration dates than from a second location where the one or more items are less likely to remain unused past their expiration dates.

Implementations of the current subject matter can include methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a personal area network, a peer-to-peer network, a mesh network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to medical inventory including pharmaceuticals, equipment, and supplies, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a medical inventory management system, in accordance with some example embodiments;

FIG. 2A depicts a schematic diagram illustrating an example of a soon-to-expire (STE) analysis model, in accordance with some example embodiments;

FIG. 2B depicts a schematic diagram illustrating another example of a soon-to-expire (STE) analysis model, in accordance with some example embodiments;

FIG. 3 depicts a flowchart illustrating an example of a process for medical inventory management with soon-to-expire (STE) analysis, in accordance with some example embodiments;

FIG. 4A depicts a schematic diagram illustrating the logic flow of an example of a soon-to-expire (STE) analysis model, in accordance with some example embodiments;

FIG. 4B depicts a schematic diagram illustrating the logic flow of another example of a soon-to-expire (STE) analysis model, in accordance with some example embodiments; and

FIG. 5 depicts a block diagram illustrating an example of a computing system, in accordance with some example embodiments.

When practical, similar reference numbers denote similar structures, features, or elements.

### DETAILED DESCRIPTION

One primary objective of medical inventory management software applications is to reduce operational costs and waste through stocking, distribution, consumption, and disposal of various pharmaceuticals, equipment, and other supplies. For example, the medical inventory management software application deployed at a healthcare facility may perform analysis to predict, within the current inventory of medical supplies at the location, supplies that may expire or be moved to another location to be used within a certain period of time. In some implementations, once an item has been moved or identified for a move, the location or item may be excluded from further soon-to-expire analysis. The exclusion may be limited in time (e.g., 10 days after move) or event limited (e.g., until an inventory update event such as restock). Medical supplies identified as soon-to-expire (STE) are those that may be expected to expire within a given period of time. These soon-to-expire items are typically removed from storage before being destroyed or destocked from its current location. In the latter case, the destocked medical supplies may either be restocked at a different location or returned for at least a partial refund.

As used herein, an item may expire (and be assigned an expiration date) based on how long the item is expected to remain effective and/or safe for use. It should be appreciated that the expiration timeframe may vary depending on the item. Some items, such as certain drugs that are compounded and/or repackaged locally (e.g., at a hospital, pharmacy, or another licensed facility), may be “short-dated”, meaning that these items have a very short expiration timeframe (e.g., hours, days, or weeks, and not months or years) during which the items are deemed soon-to-expire. In other cases, the item may be very expensive, in which case the item may need to be moved and stocked at a location where the likelihood of the item being used prior to its expiration date is maximized. Contrastingly, for a lower cost item, the cost of transferring and restocking the item at a different location may outweigh that of the item itself, in which case the timeframe during which the item is deemed soon-to-expire and moved to a new location may be shortened and be closer to the expiration date of the item. For items that are in short supply, the need to avoid waste due to expiration may override the cost of the items. As such, the timeframe during which an item that is in short supply is deemed soon-to-expire and moved to a new location may be shorter than that for an item for which there is more ample supply.

Nevertheless, conventional medical inventory management software applications are inadequate for a number of reasons. For example, conventional medical inventory management software applications perform soon-to-expire (STE) analysis based on the earliest expiration date of the current inventory at a particular location even though this value is often incorrect because clinicians are not obligated to remove stock with the earliest expiration date while the expiration dates of the current inventory are not always validated and updated as a part of the restocking workflow. Moreover, the results of the soon-to-expire (STE) analysis trigger reactive measures, such as the destruction of expired medical supplies and medical supplies that are too near their expiration dates to be restocked elsewhere, that thwart efforts to reduce operational costs and waste.

In some example embodiments, an inventory controller may perform soon-to-expire (STE) analysis by applying a soon-to-expire (STE) analysis model trained to identify one or more items, such as medications, equipment, and other supplies, that are unlikely to be used in its current location. The inventory controller may apply various implementations of the soon-to-expire (STE) analysis model including a heuristic based model and a hybrid model that combines one or more heuristic models and machine learning models. In some cases, the soon-to-expire (STE) analysis model may be trained based on historical data points associated with a medical facility and/or one or more similar medical facilities. The historical data points ingested by the soon-to-expire (STE) analysis model may be associated with one or more dispensing events, location, and inventory levels for the various items stocked at a medical facility. Examples of such data points may include, for a particular item stocked at the medical facility, a quantity of the item removed during a current time period, an inventory level of the item (e.g., as measured in value) during the current time period, a quantity of the item consumed during a previous time period, a distance to an earliest expiration date associated with the item during the current time period, and/or the like.

In some example embodiments, the inventory controller applying the soon-to-expire (STE) analysis model may identify, in advance, one or more items that are unlikely to be used in its current location. As such, upon determining that an item is unlikely to be used in its current location, the inventory controller may perform a variety of corrective actions to prevent the item from becoming outdated at its current location. For example, the inventory controller may apply the soon-to-expire (STE) analysis model to determine that a particular item is more likely to remain unused past its expiration date at a first location than at a second location, in which case the inventory controller may reallocate the item from the first location to the second location. Alternatively and/or additionally, the inventory controller may prioritize the dispensing of the item from a first location where the item is more likely to remain unused past its expiration date than from a second location where the item is less likely to remain unused. In some cases, the first location may be prioritized as a dispensing location if a larger quantity of the item present at the first location is likely to remain unused past its expiration date than at the second location. Furthermore, in some instances, the inventory controller may apply the soon-to-expire (STE) model to determine the quantity of the item that is likely to remain unused and adjust the quantity of the item that is ordered for restocking accordingly, as well as possibly adjusting the reorder point and maximum (or minimum) quantity of that item in the location for future restocking, or even recommending removal of the item from an inventory location altogether.

In some example embodiments, the soon-to-expire (STE) analysis model may be trained to recognize the nexus between individual items and different stocking locations. In particular, the item and the location stocking the item may exhibit a certain combination of characteristics that affect the likelihood of the item remaining unused past its expiration date at the location. Accordingly, the soon-to-expire (STE) analysis model may be trained to identify this combination of characteristics in order to identify the items that are likely to remain unused past their expiration date at a particular stocking location and the quantities thereof. In this context, the term “location,” which is used interchangeably with the term “stocking location,” may refer a medical setting at any level of granularity. For example, a location stocking an item may be a specific device storing the item (e.g., a dispensing cabinet, shelf, and/or the like), a building (or portion of a building) at which the device is located, a department, a room within a department, a unit, or a care area of a facility associated with the item, the facility itself, a geographic region of the facility, a network that includes the facility along with one or more other facilities, and/or the like.

The likelihood of an item remaining unused past its expiration date at its current stocking location may vary over time due to changes in a variety of factors including, for example, the velocity at which the item is used at the location, the variety of items stocked at the location, the cost of the item, the expiration pattern of the item, the movement pattern of the item, and/or the like. Accordingly, in some example embodiments, the inventory controller may subject the soon-to-expire (STE) analysis model to periodic updates in order to accommodate changes in factors that impact the soon-to-expire (STE) analysis of the items stocked at a particular medical facility. For example, in some cases, when the soon-to-expire (STE) analysis model was trained at a first time to, the soon-to-expire (STE) analysis model may be updated at one or more successive time points thereafter. At a second time t1, for instance, the soon-to-expire (STE) analysis model may be updated by being trained based on data points from between the first time to and the second time t1.

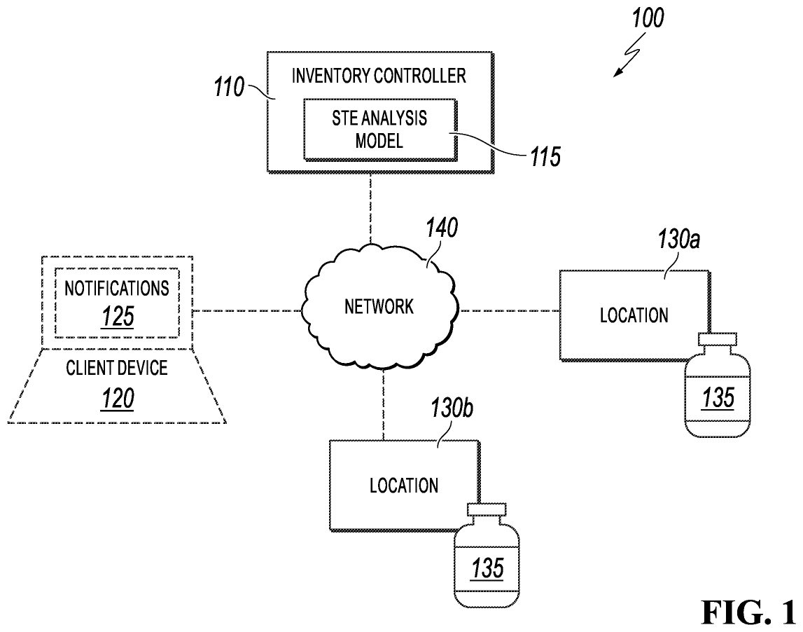

FIG. 1 depicts a system diagram illustrating an example of a medical inventory management system 100, in accordance with some example embodiments. Referring to FIG. 1, the medical inventory management system 100 may include an inventory controller 110, a client device 120, and one or more medical locations 130. As shown in FIG. 1, the inventory controller 110, the client device 120, and the one or more medical locations 130 may be communicatively coupled via a network 140. The client device 120 may be a specifically configured processor-based device including, for example, a point of care unit (PCU), a smartphone, a tablet computer, a wearable apparatus, a desktop computer, a laptop computer, a workstation, and/or the like. Meanwhile, the network 140 may be a wired and/or wireless network including, for example, a public land mobile network (PLMN), a local area network (LAN), a virtual local area network (VLAN), a wide area network (WAN), the Internet, and/or the like.

In some example embodiments, the inventory controller 110 may apply a soon-to-expire (STE) analysis model 115 in order to perform soon-to-expire (STE) analysis for an item 135 stocked at the one or more locations 130. For example, the inventory controller 110 may apply the soon-to-expire (STE) analysis model 115 to determine the likelihood of the item 135 stocked at the one or more locations 130 remaining unused past its expiration date. Alternatively and/or additionally, the inventory controller 110 may apply the soon-to-expire (STE) analysis model 115 to determine a quantity of the item 135 stocked at the one or more locations 130 that will remain unused past its expiration date.

In some example embodiments, the inventory controller 110 may apply the soon-to-expire (STE) analysis model to determine, in advance, that the item 135 is likely to remain unused in its current location and trigger a variety of corrective actions to prevent the item 135 from becoming outdated at its current location or, in some cases, to ensure that the item 135 is destocked early enough before its expiration to be restocked elsewhere. For example, when the inventory controller 110 determines that the item 135 is likely to remain unused past its expiration date at a first location 130a but will be used prior to its expiration date at a second location 130b, the inventory controller 110 may reallocate the item 135 from the first location 130a to the second location 130b. In some cases, reallocating the item 135 from the first location 130a to the second location 130b may include transferring the item 135 from one distributed dispensing location (e.g., a first dispensing cabinet) to another distributed dispensing location (e.g., a second dispensing cabinet at a same or different facility or portion of the facility). In other instances, reallocating the item 135 from the first location 130a to the second location 130b may include transferring the item 135 from a distributed dispensing location (e.g., a first dispensing cabinet) to a central dispensing location (e.g., a central pharmacy, a warehouse, and/or the like).

In some example embodiments, the inventory controller 110 may generate one or more electronic records associated with the item being reallocated from the first location 130a to the second location 130b. For example, reallocating the item 135 from the first location 130a to the second location 130b may include transferring the custody of the item 135 from a first user associated with the first location 130a to a second user associated with the second location 130b. Accordingly, the inventory controller 110 may generate one or more electronic records to document the chain of custody associated with the item 135. In some instances, the item 135 may be subject to certain regulatory requirements that necessitate a certain chain of custody. Accordingly, the reallocation from the first location 130a to the second location 130b may further include an intermediary location, such as a pharmacy.

In cases where the item 135 is stocked at multiple locations, the inventory controller 110 may cause the item 135 to be dispensed from the first location 130a if the item 135 is more likely to remain unused past its expiration date at the first location 130a than the second location 130b and/or if a larger quantity of the item 135 likely to remain unused past its expiration date is present at the first location 130a than the second location 130b. For example, consider when the first location 130a is a first drawer of an automated dispensing cabinet and the second location 130b is a second drawer of the automated dispensing cabinet. Instances of the item 135 may be stored in both locations. When a clinician requests a dispense of the item 135, the inventory controller 110 may apply the STE model (or review information generated by the STE model) to determine which location to release to dispense the item 135. The inventory controller 110 may authorize dispense from the location identified as most likely to expire closest to the current date. In some instances, the inventory controller 110 may apply the soon-to-expire (STE) analysis model 115 to determine the quantity of the item 135 that is likely to remain unused and adjust the quantity of the item 135 that is ordered for restocking at the first location 130a and/or the second location 130b accordingly.

In some example embodiments, upon identifying the item 135 as being likely to remain unused past its expiration date at the one or more locations 130, the inventory controller 110 may send, to the client device 120 associated with the one or more locations 130, one or more corresponding notifications 125. For example, the inventory controller 110 may send, to the first location 130a, the notifications 125 with instructions to review expiration dates of the item 135 stocked at the first location 130a, destock the item 135 from the first location 130a, and/or remove the item 135 from the first location 130a. In some cases, the instructions may further specify a certain quantity of the item 135 for destocking and removal from the first location 130a and for transfer and stocking at the second location 130b. In some cases, the inventory controller 110 may send the notifications 125 at specific times in order to ensure that the item 135 is destocked and removed from the first location 130a early enough for it to be restocked and consumed at the second location 130b before its expiration date. If, for example, the item 135 is a high-cost item due to expire in a short period of time but is stocked at the first location 130a, which has a history of infrequent dispensing activities, the inventory controller 110 may send the notifications 125 at an earlier time and/or at a higher frequency.

In some example embodiments, the soon-to-expire (STE) analysis model 115 may be trained to recognize the nexus between individual items, such as the item 135, and the different stocking locations 130. In particular, the item 135 and the one or more locations 130 stocking the item 135 may exhibit a certain combination of characteristics that affect the likelihood of the item 135 remaining unused past its expiration date. For example, the item 135 stocked at the first location 130a may exhibit a different set of characteristics than the item 135 stocked at the second location 130b. Examples of these characteristics include the velocity at which the item 135 is used at each of the locations 130, the cost of the item 135, the expiration pattern of the item 135, the movement pattern of the item 135, and/or the like. When trained, the soon-to-expire (STE) analysis model 115 may be capable of identifying the item 135 as likely to expire at the first location 130a with sufficient time, for example, for the item 135 to be destocked from the first location 130a and transferred to the second location 130b where the item 135 can be consumed prior to its expiration date, thus realizing significant reduction in operational costs and waste.

In some example embodiments, the inventory controller 110 may periodically update the soon-to-expire (STE) analysis model 115 at least because the likelihood of the item 135 remaining unused past its expiration date at its current stocking location may vary over time due to changes in factors such as the velocity at which the item is used at the location, changes in the variety of items stocked at the location, the cost of the item, the expiration pattern of the item, the movement pattern of the item, and/or the like. For example, where the soon-to-expire (STE) analysis model 115 was trained at a first time to, the inventory controller 110 may subsequently update the soon-to-expire (STE) analysis model 115 at a second time t1 by training the soon-to-expire (STE) analysis model 115 based on data points from between the first time to and the second time t1.

Changes in one or more of the aforementioned factors may be attributable to simple causes, such as a change the packaging, manufacture, storage requirement, and/or other characteristics of the item, and can therefore be a common occurrence. For example, the item 135 may be associated with a shorter expiration date if the item 135 is a compounded item, a repackaged item, and/or an item requiring special storage (e.g., refrigeration and/or the like). Updating the soon-to-expire (STE) analysis model 115 through periodic retraining of the soon-to-expire (STE) analysis model 115 may enable the soon-to-expire (STE) analysis model 115 to respond to the aforementioned changes and maintain the accuracy of its soon-to-expire (STE) analysis.

In some example embodiments, the soon-to-expire (STE) analysis model 115 may be trained to perform soon-to-expire (STE) analysis for the item 135 based on a variety of data points including, for example, cost of the item 135, a velocity or usage rate of the item 135, a quantity of the item 135 removed, a current inventory level of the item 135, a quantity of the item 135 consumed during a previous time period, a distance to an earliest expiration date associated with the current stock of the item 135, and/or the like. Other data points that may be incorporated into the soon-to-expire (STE) analysis of the item 135 may include its packaging, storage requirements, whether the item was a custom compound, or the like.

At least some of the aforementioned data points may be represented as a categorical value. For example, the cost of the item 135 may be represented as a first binary value indicating whether the item 135 is a high cost item or a low cost item, the current inventory level of the item 135 may be represented as a second binary value indicating whether the item 135 is associated with a low inventory value or a high inventory value, and the usage rate of the item 135 may be represented as a third binary value indicating whether the item 135 has a low usage rate or a high usage rate. Meanwhile, the quantity of the item 135 removed, for example, due to being outdated or for destocking, may be represented as a category selected from multiple categories of removal percentages (e.g., a ratio of a first quantity of the item 135 removed and a second quantity of the item 135 in the inventory at the beginning of the time period).

The categorical values representing the aforementioned data points, such as the thresholds for low or high unit cost, low or high inventory dollar value, and low or high usage rate, as well as the percentiles for the different categories of removal percentages, may be determined based on the corresponding error (e.g., mean absolute error (MAE) or a different error metric). For example, in some cases, the inventory controller 110 may determine, as a part of training the soon-to-expire (STE) analysis model 115, the threshold separating a high value category and a low value category based on the corresponding historical data from each of the one or more locations 130. Accordingly, the high versus low threshold, for example, may be identified as a percentile of the existing dataset (e.g., 75^{th}, 80^{th}, or 85^{th}) associated with a minimum error (e.g., mean absolute error (MAE) or a different error metric).

In some implementations, when the system identifies an item 135 or location 130a, 130b as containing soon-to-expire items 135 for reallocation or destocking, the system may cause the location to secure the location until the reallocation or destocking occurs. For example, if a first location of an automated dispensing cabinet (e.g., drawer or pocket or bin), includes an item that is deemed to be expiring soon (e.g., within a predetermined number of days of a predicted soon to expire date; within a predetermined expiration confidence range; etc.), the automated dispensing cabinet may be configured to prevent any further dispensing from the first location unless the access request is made by a clinician performing a destock or reallocation. The access request may be identified based on credentials or other user identifying information provided by the clinician accessing the automated dispensing cabinet. The access request may be based on an action selected at the automated dispensing cabinet. For example, a clinician may activate a control element on a user interface to activate a destock or reallocation mode. Requests for unlocking or locking locations while in this mode may be distinguishable from dispense requests for a specific patient.

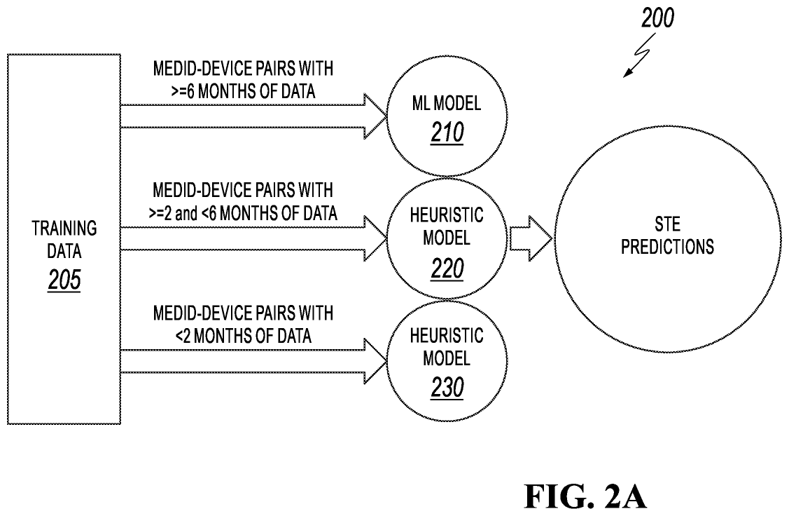

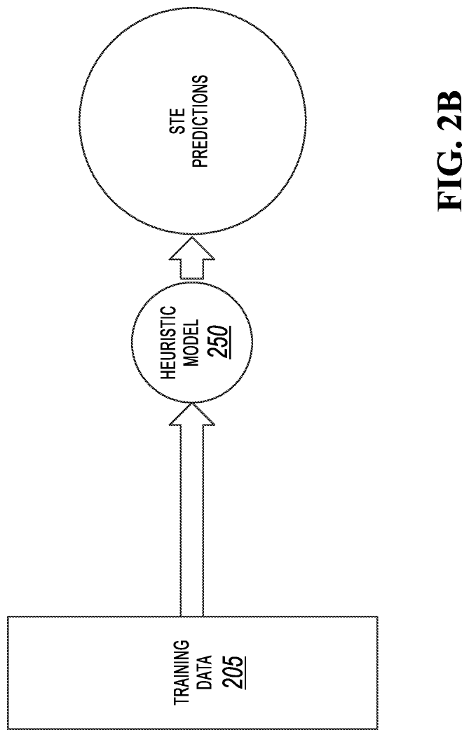

In some example embodiments, the soon-to-expire (STE) analysis model 115 may be implemented as a heuristic model or a hybrid model that combines one or more heuristic models and machine learning models. FIG. 2A depicts a schematic diagram illustrating an example of the soon-to-expire (STE) analysis model 115 implemented as a hybrid model 200 while FIG. 2B depicts another example of the soon-to-expire (STE) analysis model 115 implemented as a heuristic model 250. In some cases, the inventory controller 110 may select an implementation of the soon-to-expire (STE) analysis model 115 that is more suitable for the one or more locations 130, for example, by being associated with a lower prediction error (e.g., mean absolute error (MAE) or a different error metric). For example, in cases where the soon-to-expire (STE) analysis model 115 is implemented as a heuristic model, the inventory controller 110 may identify, from a selection of heuristic models incorporating different combinations of data points and categorical values, one having the lowest prediction error. Table 1 below depicts some examples of heuristic models and the corresponding data points.

TABLE 1

| Model | Data Points |
|---|---|
| Model 1 | Item Unit Value (low/high) + Item Usage Rate |
| Model 2 | Item Value (low/high) + Item Usage Rate |
| Model 3 | Item Unit Value (low/high) + Distance to Earliest |
| | Expiration Date |
| Model 4 | Item Value (low/medium/high) + Item Usage Rate |

The inventory controller 110 may assess the models by providing a data for a controlled group of items to each model, comparing the model prediction to an actual or desired output for each of the items, and, based on the comparison, select the model having the highest rate of success in predicting the actual or desired outputs.

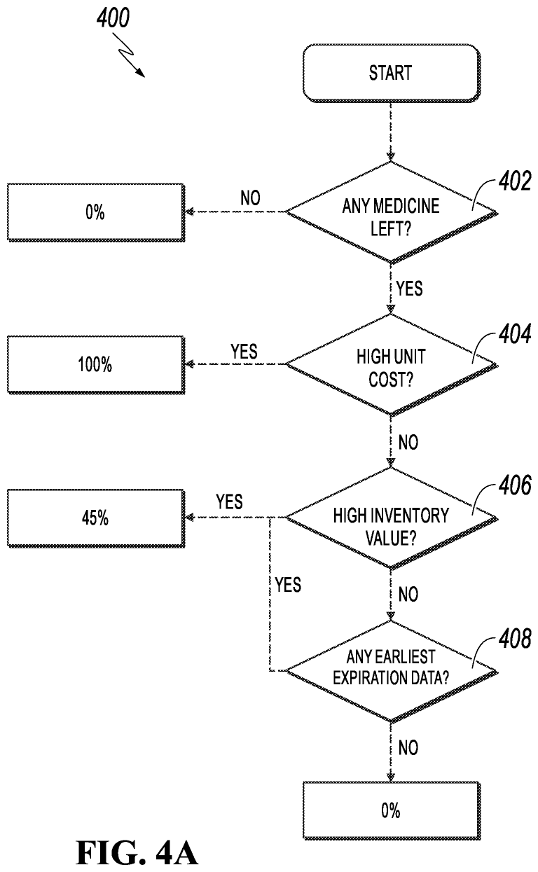

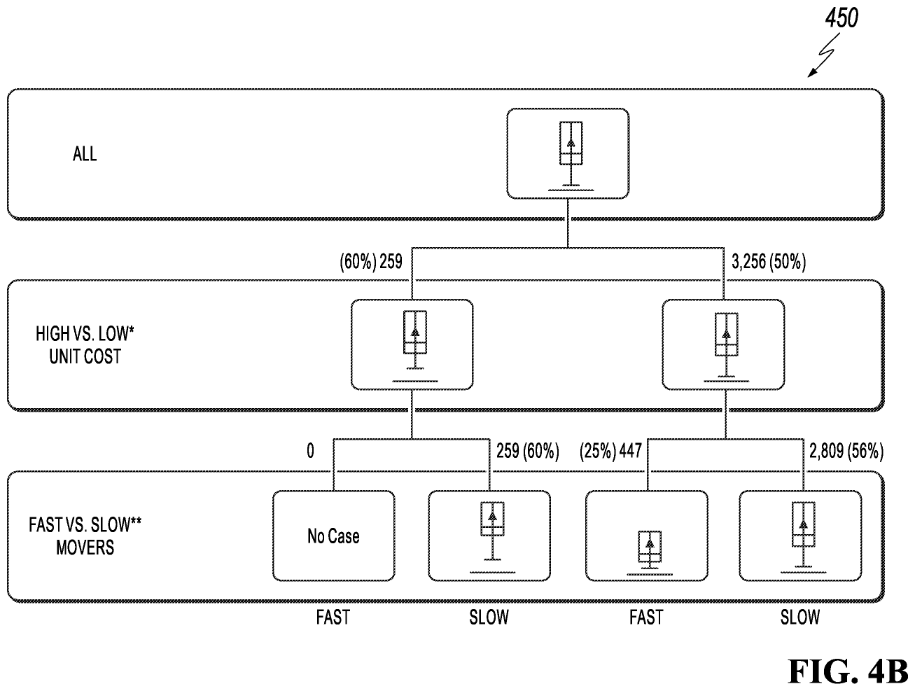

FIG. 4A depicts a schematic diagram illustrating the logic flow 400 of one example of the soon-to-expire (STE) analysis model 115 implemented as a heuristic model that determines a percentage of the current stock of the item 135 at the one or more locations 130 that is likely to remain unused past its expiration date. In the example shown in FIG. 4A, the logic flow 400 may include a combination of factors, such as current inventory level, unit cost, inventory value, and earliest expiration date, to determine the percentage of the current stock of the item 135 likely to remain unused past its expiration date. As noted, this combination of factors may be selected as a part of training the soon-to-expire (STE) analysis model 115 based on this combination of factors being associated with the least error (e.g., as measured by a mean absolute error (MAE) or a different error metric). Moreover, as shown in FIG. 4A, the soon-to-expire (STE) analysis model 115 may apply one or more of the aforementioned categorical values associated with the item 135. It should be appreciated that the inventory controller 110 may perform one or more corrective actions based on the percentage of the item 135 likely to remain unused including, for example, the destocking and/or reallocation of a corresponding quantity of the item 135.

In the example of the logic flow 400 shown in FIG. 4A, at block 402, the soon-to-expire (STE) analysis model 115 may first determine whether any quantity of the item 135 remain at the one or more locations 130. If some quantities of the item 135 remain at the one or more locations 130, the soon-to-expire (STE) analysis model 115 may next determine, at block 404, whether the item 135 is a high-cost item or a low-cost item. As noted earlier, the threshold for whether the item 135 is categorized as a high unit cost item, or a low unit cost item may be determined as a part of training the soon-to-expire (STE) analysis model 115.

Moreover, in the example shown in FIG. 4A, if the item 135 is determined to be a high unit cost item, then the soon-to-expire (STE) analysis model 115 may determine that 100% of the current stock of the item 135 is likely to remain unused past its expiration date. Alternatively, if the item 135 is determined to be a low unit cost item, the soon-to-expire (STE) analysis may continue with the soon-to-expire (STE) analysis model 115 determining, at block 406, whether the item 135 is associated with a high inventory value (e.g., unit cost of item multiplied by the number of items in inventory at a location; holding cost (e.g., hazardous, risky, or divertible drugs) multiplied by the number of items in inventory at a location; cost to replenish or replace (e.g., time to re-compound or order) an item multiplied by the number of items in inventory at a location). In the event the item 135 is associated with a high inventory value, the soon-to-expire (STE) analysis model 115 may determine that 45% of the current stock of the item 135 is likely to remain unused past its expiration date. However, if the item 135 is not associated with a high inventory value, the soon-to-expire (STE) analysis model 115 may determine, at block 408, that 45% of the current stock of the item 135 is likely to remain unused past its expiration date if the current stock of the item 135 is associated with an earliest expiration date. Contrastingly, if the item 135 is not associated with a high inventory value and has no earliest expiration date, then the soon-to-expire (STE) analysis model 115 may determine that 0% of the current stock of the item 135 is likely to remain unused past its expiration date and thereby indicating no need for inventory adjustment(s).

FIG. 4B depicts a schematic diagram illustrating the logic flow 450 of another example of the soon-to-expire (STE) analysis model 115, in accordance with some example embodiments. The example of the soon-to-expire (STE) analysis model 115 shown in FIG. 4B is configured to determine a removal percentage of the item 135 stocked at one or more locations 130 based on a combination of factors that include the unit cost of the item 135 and the velocity of the item 135. In some cases, the inventory controller 110 may perform one or more corrective actions based on this removal percentage including, for example, the destocking and/or reallocation of a corresponding quantity of the item 135. Again, it should be appreciated that this combination of factors may be selected during the training of the soon-to-expire (STE) analysis model 115 based on the corresponding error (e.g., mean absolute error (MAE) or a different error metric). Moreover, each of the factors may be evaluated as categorical values whose thresholds (e.g., for high unit value and low unit value, fast moving and slow moving, and/or the like) are selected as part of training the soon-to-expire (STE) analysis model 115 based on the corresponding error (e.g., mean absolute error (MAE) or a different error metric).

Referring again to FIG. 4B, the soon-to-expire (STE) analysis model 115 implementing the logic flow 450 may first determine whether the item 135 is a high unit cost item or a low unit cost item before determining whether the item is a fast-moving item or a slow-moving item. As shown in FIG. 4B, if the item 135 is determined to be a high unit cost item that is also fast moving, the soon-to-expire (STE) analysis model 115 may identify 0% of the current stock of the item 135 for removal. Contrastingly, if the item 135 is determined to be a slow-moving high unit cost item, the soon-to-expire (STE) analysis model 115 may identify that 60% of the current stock of the item 135 for removal. In the event the item 135 is a fast-moving low unit cost item, the soon-to-expire (STE) analysis model 115 may identify 25% of the current stock of the item 135 for removal whereas 56% of the current stock of the item 135 may be identified for removal if the item 135 is determined to be a slow-moving low unit cost item.

In cases where the soon-to-expire (STE) analysis model 115 is implemented as a hybrid model that combines one or more heuristic models and machine learning models, the inventory controller 110 may train and deploy the one or more heuristic models and the machine learning models based on the quantity of the data available for training the soon-to-expire (STE) analysis model 115. In some cases, the training data used for training the soon-to-expire (STE) analysis model 115 may be location specific. For example, the soon-to-expire (STE) analysis model 115 may be trained based on training data that includes historical data from the first location 130a. Accordingly, the soon-to-expire (STE) analysis model 115 may be trained to recognize the combination of characteristics that affect the likelihood of the item 135 remaining unused past its expiration date at the first location 130a. Moreover, once trained, the soon-to-expire (STE) analysis model 115 may be applied to current data from the first location 130a to determine whether the current stock of the item 135 at the first location 130a is likely to remain unused past its expiration date. In cases where the second location 130b is sufficiently similar to the first location 130a, the soon-to-expire (STE) analysis model 115 trained on data from the first location 130a may also be applied to determine whether the current stock of the item 135 at the second location 130b is likely to remain unused past its expiration date.

In some implementations, the system may determine that based on the types of items or other local clinical dispensing factors, the evaluation of slow versus fast movers is more efficient or accurate at determining soon-to-expire status for an item. In such instances, the system may evaluate not just different models, but different modelling pipelines to identify an optimally accurate configuration for the site. The optimization may consider not just accuracy but also time and other resources needed to generate a soon-to-expire status. For example, if a pipeline that considers item velocity (fast v. slow movers) and then unit cost generates soon-to-expire status for test items at a rate of 1 (e.g., 1 second) or using x amount of resources (e.g., network communication, processing cycles, memory, etc.). If a different configuration takes less time or uses less amount, then the alternate configuration would be selected by the system.

To further illustrate, the example of the hybrid model 200 shown in FIG. 2A includes a machine learning model 210, a first heuristic model 220, and a second heuristic model 230, each of which having a different requirement for the quantity of training data. Accordingly, in cases where the quantity of the training data available satisfies a first threshold (e.g., more than 6 months of historical data is available), the hybrid model 200 may be trained by training the machine learning model 210 based on the training data. Where the quantity of the training data fails to satisfy the first threshold but satisfies a second threshold (e.g., less than 6 months but more than 2 months of data is available), the hybrid model 200 may be trained by training the first heuristic model 220 based on the training data. In cases where the quantity of the training data fails to satisfy the second threshold (e.g., less than 2 months of data is available), the hybrid model 200 may be trained by training the second heuristic model 230 based on the training data.

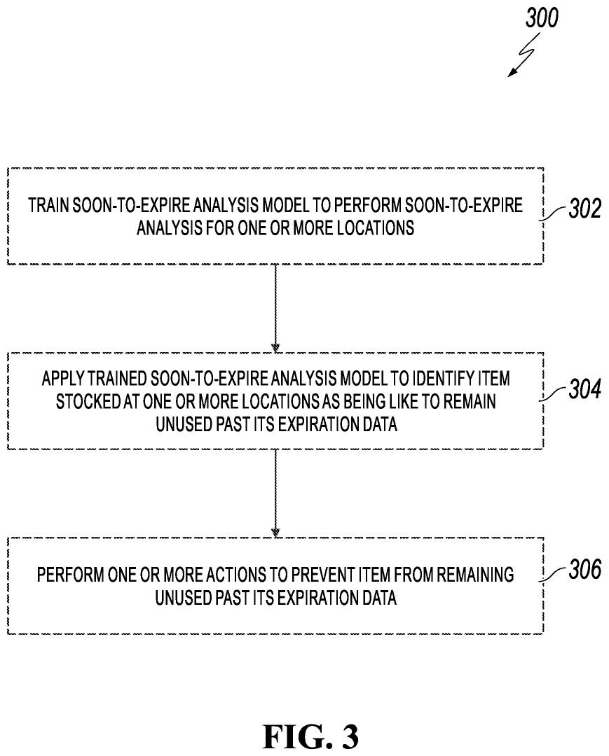

FIG. 3 depicts a flowchart illustrating an example of a process 300 for medical inventory management with soon-to-expire (STE) analysis, in accordance with some example embodiments. Referring to FIGS. 1-3, the process 300 may be performed by the inventory controller 110 applying the soon-to-expire (STE) analysis model 115 to determine, for example, whether the item 135 stocked at the one or more locations 130 is likely to remain unused past its expiration date at its current location.

At 302, the inventory controller 110 may train a soon-to-expire (STE) analysis model to perform soon-to-expire (STE) analysis for one or more locations. In some example embodiments, the inventory controller 110 may train the soon-to-expire (STE) analysis model 115 to perform soon-to-expire (STE) analysis for the one or more locations 130. The soon-to-expire (STE) analysis model 115 may be implemented as a heuristic model (e.g., the heuristic model 250) or a hybrid model (e.g., the hybrid model 200) that combines one or more machine learning models and heuristic models. Moreover, the soon-to-expire (STE) analysis model 115 may be trained based on historical data points associated with the one or more locations 130. Examples of such data points include, for each item stocked at the one or more locations 130 such as the item 135, a quantity of the item removed during a current time period, an inventory level of the item (e.g., as measured in value) during the current time period, a quantity of the item consumed during a previous time period, a distance to an earliest expiration date associated with the item during the current time period, and/or the like. Through training, the soon-to-expire (STE) analysis model 115 may recognize the combination of characteristics that affect the likelihood of the item 135 remaining unused past its expiration date at each of the one or more locations. Examples of these characteristics include the velocity at which the item 135 is used at each of the locations 130, the cost of the item 135, the expiration pattern of the item 135, the movement pattern of the item 135, and/or the like.

As noted, the soon-to-expire (STE) analysis model 115 may be implemented as one or more heuristic models and/or machine learning models. Thus, it should be appreciated that one or more aspects of the artificial intelligence described may be implemented in whole or in part by a model, including a machine learning model. The training that the model is subjected to may be supervised, unsupervised, reinforced, or a hybrid approach whereby multiple learning techniques are employed to generate the model. Training the model may include obtaining a set of training data and adjusting characteristics of the model to obtain a desired model output. For example, three characteristics may be associated with a desired device state. In such instance, the training may include receiving the three characteristics as inputs to the model and adjusting the characteristics of the model such that for each set of three characteristics, the output device state matches the desired device state associated with the training data. In some cases, the training may be dynamic, meaning that the system may update the model using a set of events with detectable properties of the events used to adjust the model.

In some cases, the soon-to-expire (STE) analysis model 115 may be an equation, an artificial neural network, a recurrent neural network, a convolutional neural network, a decision tree, and/or another machine readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds or limits for evaluating an output value, or criterion for selecting from a set of equations.

Once the soon-to-expire (STE) analysis model 115 is trained, retraining may be included to refine or update the model to reflect additional data or specific operational conditions. For example, as noted, the inventory controller 110 may subject the soon-to-expire (STE) analysis model to periodic updates in order to accommodate changes in factors that impact the soon-to-expire (STE) analysis of the items stocked at a particular medical facility. The retraining may be based on one or more signals detected by a device described herein or as part of a method described herein. Upon detection of the designated signals, the system may activate a training process to adjust the soon-to-expire (STE) analysis model 115 as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

At 304, the inventory controller 110 may apply the trained soon-to-expire (STE) analysis model to identify an item stocked at the one or more locations as being likely to remain unused past its expiration date. The trained model may look at an item's unit cost, inventory quantity, days to its earliest expiration date, and its usage rate and provide a soon-to-expire amount for that item. For example, the inventory controller 110 may apply the trained soon-to-expire (STE) analysis model 115 to identify the item 135 as being likely to remain unused past its expiration date at the one or more locations 130. The inventory controller 110 may apply the soon-to-expire (STE) analysis model 115 on a periodic basis or upon detecting a change in the inventory of one or more items such as the item 135. Moreover, the inventory controller 110 may apply the soon-to-expire (STE) analysis model 115 to identify items that are likely to remain unused past its expiration date at an individual location or at a group of locations, such as locations within a facility, a same geographic region, a same network. To apply the soon-to-expire (STE) analysis model 115, the inventory controller 110 may provide, for ingestion by the soon-to-expire (STE) analysis model 115, information associated with at least a portion of the items currently in stock at the one or more locations 130 such as current inventory level, unit cost, inventory value, and earliest expiration date. The logic of the soon-to-expire (STE) analysis model 115 may be applied to process the current inventory information and generate an output identifying the items that are likely to remain unused past its expiration date at their current location. In some cases, the output of the soon-to-expire (STE) analysis model 115 may include a listing of items ranked by their respective likelihood of expiring at their current locations such that one or more corrective actions to prevent the expiration of these items may be performed based on the listing.

At 306, the inventory controller 110 may perform one or more corrective actions to prevent the item from remaining unused past its expiration date. When trained, the soon-to-expire (STE) analysis model 115 may be applied to identify the item 135 as likely to expire at, for example, the first location 130a with sufficient time for corrective actions, such as destocking the item 135 from the first location 130a and transferring to the second location 130b, to ensure that the item 135 can be consumed prior to its expiration date. For example, upon identifying the item 135 as being more likely to remain unused past its expiration date at the first location 130a than at a second location 130b, the inventory controller 110 may reallocate the item 135 from the first location 130a to the second location 130b. Alternatively and/or additionally, the inventory controller 110 may prioritize the dispensing of the item 135 from the first location 130a over the second location 130b if the item 135 is more likely to remain unused past its expiration date at the first location 130a than at the second location 130b. In some cases, the inventory controller 110 may apply the trained soon-to-expire (STE) analysis model 115 to determine the quantity of the item 135 likely to remain unused at each of the first location 130a and the second location 130b. Where a larger quantity of the item 135 is likely to remain unused is present at the first location 130a than at the second location 130b, the inventory controller 110 may trigger the reallocation and/or prioritized dispensing of a certain quantity of the item 135 from the first location 130a. In some instances, the inventory controller 110 may also adjust the quantity of the item 135 that is ordered for restocking at the first location 130a and/or the second location 130b based on the quantity of the item 135 likely to remain unused at each of the first location 130a and the second location 130b. Alternatively and/or additionally, the inventory controller 110 may adjust the reordering schedules of the item 135 and/or the maximum (or minimum) quantity of the item 135 stocked at the first location 130a and/or the second location 130b based on the quantity of the item 135 likely to remain unused at each of the first location 130a and the second location 130b. In some cases, the inventory controller 110 may even recommend removal of the item 150 from being stocked at the first location 130a and/or the second location 130b altogether.

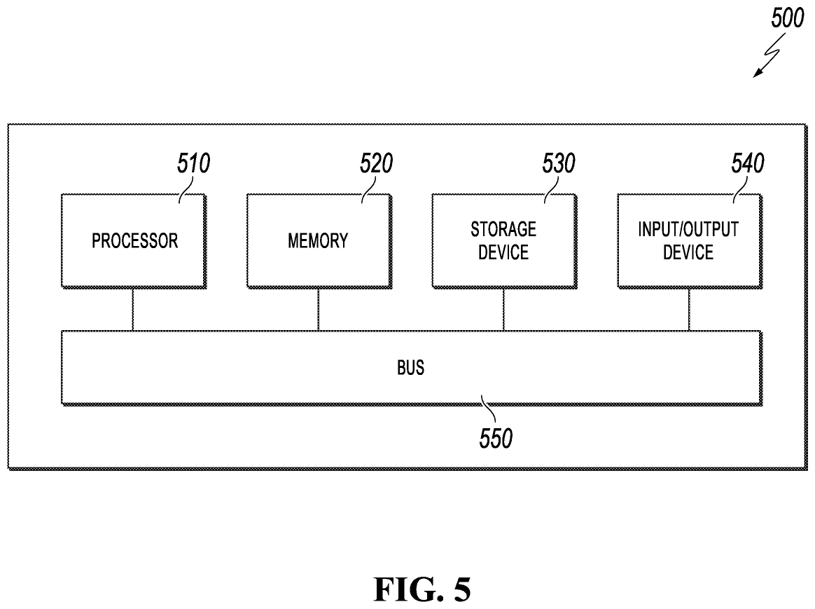

FIG. 5 depicts a block diagram illustrating a computing system 500 consistent with implementations of the current subject matter. Referring to FIGS. 1 and 5, the computing system 500 can be used to implement the inventory controller 110 and/or any components therein.

As shown in FIG. 5, the computing system 500 can include a processor 510, a memory 520, a storage device 530, and an input/output device 540. The processor 510, the memory 520, the storage device 530, and the input/output device 540 can be interconnected via a system bus 550. The processor 510 is capable of processing instructions for execution within the computing system 500. Such executed instructions can implement one or more components of, for example, the inventory controller 110. In some example embodiments, the processor 510 can be a single-threaded processor. Alternatively, the processor 510 can be a multi-threaded processor. The processor 510 is capable of processing instructions stored in the memory 520 and/or on the storage device 530 to display graphical information for a user interface provided via the input/output device 540.

The memory 520 is a computer readable medium such as volatile or non-volatile that stores information within the computing system 500. The memory 520 can store data structures representing configuration object databases, for example. The storage device 530 is capable of providing persistent storage for the computing system 500. The storage device 530 can be a floppy disk device, a hard disk device, an optical disk device, a tape device, a solid-state device, and/or any other suitable persistent storage means. The input/output device 540 provides input/output operations for the computing system 500. In some example embodiments, the input/output device 540 includes a keyboard and/or pointing device. In various implementations, the input/output device 540 includes a display unit for displaying graphical user interfaces.

According to some example embodiments, the input/output device 540 can provide input/output operations for a network device. For example, the input/output device 540 can include Ethernet ports or other networking ports to communicate with one or more wired and/or wireless networks (e.g., a local area network (LAN), a wide area network (WAN), the Internet).

In some example embodiments, the computing system 500 can be used to execute various interactive computer software applications that can be used for organization, analysis and/or storage of data in various formats. Alternatively, the computing system 500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 540. The user interface can be generated and presented to a user by the computing system 500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™,.NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

Further non-limiting aspects or implementations are set forth in the following numbered examples:

Example 1: A method comprising: under control of one or more processors, receiving training data comprising historical data related to an item; training a soon to expire analysis model using the training data to generate a trained soon to expire analysis model, the trained soon to expire analysis model configured to receive item data associated with the item and generate soon to expire prediction for one or more items associated with the item data; receiving inventory information for the item; processing at least a portion of the inventory information with the trained soon to expire analysis model to determine the item stocked at one or more locations as being likely to remain unused past a target date; and providing an output, to an inventory controller, to perform an action to prevent the item from remaining unused past the target date.

Example 2: The method of example 1, wherein the action to prevent the item from remaining unused past the expiration date comprises moving, by the inventory controller, at least a portion of the item from the one or more locations to a use location.

Example 3: The method of any one of the preceding examples, wherein determining the item stocked at the one or more locations as being likely to remain unused past the expiration date comprises comparing a categorical value to a threshold.

Example 4: The method of any one of the preceding examples, wherein the categorical value comprises one or more of: a unit cost, a usage velocity, and a moving speed of the item.

Example 5: The method of any one of the preceding examples, wherein the historical data comprises inventory changes from a plurality of locations.

Example 6: The method of any one of the preceding examples, wherein the soon to expire analysis model comprises a machine learning model and one or more heuristic models.

Example 7: The method of any one of the preceding examples, wherein training the soon to expire analysis model comprises: determining whether the historical data satisfies a first threshold; and in response to determining that the historical data satisfies the first threshold, executing the training using the machine learning model.

Example 8: The method of any one of the preceding examples, wherein training the soon to expire analysis model comprises: determining whether the historical data satisfies the first threshold; in response to determining that the historical data fails to satisfy the first threshold, determining whether the historical data satisfies a second threshold; and in response to determining that the historical data satisfies the second threshold, executing the training using one of the one or more heuristic models.

Example 9: The method of any one of the preceding examples, wherein the one or more heuristic models comprise a model defining an association between an item unit value and an item usage rate, an item value and the item usage rate, or an item unit value and a distance to earliest expiration date.

Example 10: The method of any one of the preceding examples, wherein training comprises any of a supervised training, an unsupervised training, a reinforced training, a dynamic training, or a hybrid training.

Example 11: The method of any one of the preceding examples, wherein training the soon to expire analysis model comprises: obtaining training item data; generating a first soon to expire analysis model including a first processing pipeline wherein the machine learning model receives the training item data and wherein a heuristic model receives, as a first input, at least a portion of a first output from the machine learning model; generating a second soon to expire analysis model including a second processing pipeline wherein the heuristic model receives the training item data and wherein the machine learning model receive, as a second input, at least a portion of a second output from the heuristic model; measuring resource utilization for processing at least a portion of the training item data using the first soon to expire analysis model and second soon to expire analysis model; and selecting one of the first soon to expire analysis model and second soon to expire analysis model as the soon to expire analysis model based on the resource utilization.

Example 12: The method of any one of the preceding examples, wherein the target data is one of: an expiration date for the item, a predetermined amount of time from a current date, or a scheduled inventory update date.

Example 13: The method of any one of the preceding examples, wherein a first instance of the item is available at a first location managed by the inventory controller and a second instance of the item is available a second location managed by the inventory controller, and wherein the action to prevent the first instance of the item from remaining unused past the target date comprises configuring the inventory controller to, upon receiving a request to dispense the item, dispense the item from the first location.

Example 14: A system, comprising: at least one data processor; and at least one memory storing instructions which, when executed by the at least one data processor, result in operations comprising: receiving training data comprising historical data related to an item; training a soon to expire analysis model using the training data to generate a trained soon to expire analysis model; applying the trained soon to expire analysis model to determine the item stocked at one or more locations as being likely to remain unused past an expiration date; and providing an output to perform an action to prevent the item from remaining unused past the expiration date.

Example 15: The system of examples 14, wherein the action to prevent the item from remaining unused past the expiration date comprises moving, by an inventory controller, at least a portion of the item from the one or more locations to an use location and wherein the historical data comprises inventory changes from a plurality of locations.

Example 16: The system of any one of the preceding examples, wherein determining the item stocked at the one or more locations as being likely to remain unused past the expiration date comprises comparing a categorical value to a threshold, wherein the categorical value comprises one or more of: a unit cost, a usage velocity, and a moving speed of the item.

Example 17: The system of any one of the preceding examples, wherein the soon to expire analysis model comprises a machine learning model and one or more heuristic models, wherein the one or more heuristic models comprise a model defining an association between an item unit value and an item usage rate, an item value and the item usage rate, or an item unit value and a distance to earliest expiration date.

Example 18: The system of any one of the preceding examples, wherein training the soon to expire analysis model comprises: determining whether the historical data satisfies a first threshold; and in response to determining that the historical data satisfies the first threshold, executing the training using the machine learning model; or in response to determining that the historical data fails to satisfy the first threshold, determining whether the historical data satisfies a second threshold; and in response to determining that the historical data satisfies the second threshold, executing the training using one of the one or more heuristic models.

Example 19: The system of any one of the preceding examples, wherein training comprises any of a supervised training, an unsupervised training, a reinforced training, a dynamic training, or a hybrid training.

Example 20: A non-transitory computer-readable medium storing instructions, which when executed by at least one data processor, result in operations comprising: receiving training data comprising historical data related to an item; training a soon to expire analysis model using the training data to generate a trained soon to expire analysis model; applying the trained soon to expire analysis model to determine the item stocked at one or more locations as being likely to remain unused past an expiration date; and providing an output to perform an action to prevent the item from remaining unused past the expiration date.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method comprising:
under control of one or more processors,
receiving training data comprising historical data related to an item;
training a soon to expire analysis model using the training data to generate a trained soon to expire analysis model, the trained soon to expire analysis model configured to receive item data associated with the item and generate soon to expire prediction for one or more items associated with the item data;
receiving inventory information for the item;
processing at least a portion of the inventory information with the trained soon to expire analysis model to determine the item stocked at one or more locations as being likely to remain unused past a target date; and
providing an output, to an inventory controller, to perform an action to prevent the item from remaining unused past the target date.

2. The method of claim 1, wherein the action to prevent the item from remaining unused past the expiration date comprises moving, by the inventory controller, at least a portion of the item from the one or more locations to a use location.

3. The method of claim 1, wherein determining the item stocked at the one or more locations as being likely to remain unused past the expiration date comprises comparing a categorical value to a threshold.

4. The method of claim 3, wherein the categorical value comprises one or more of: a unit cost, a usage velocity, and a moving speed of the item.

5. The method of claim 1, wherein the historical data comprises inventory changes from a plurality of locations.

6. The method of claim 1, wherein the soon to expire analysis model comprises a machine learning model and one or more heuristic models.

7. The method of claim 6, wherein training the soon to expire analysis model comprises:
determining whether the historical data satisfies a first threshold; and
in response to determining that the historical data satisfies the first threshold, executing the training using the machine learning model.

8. The method of claim 7, wherein training the soon to expire analysis model comprises:
determining whether the historical data satisfies the first threshold;
in response to determining that the historical data fails to satisfy the first threshold, determining whether the historical data satisfies a second threshold; and
in response to determining that the historical data satisfies the second threshold, executing the training using one of the one or more heuristic models.

9. The method of claim 6, wherein the one or more heuristic models comprise a model defining an association between an item unit value and an item usage rate, an item value and the item usage rate, or an item unit value and a distance to earliest expiration date.

10. The method of claim 1, wherein training comprises any of a supervised training, an unsupervised training, a reinforced training, a dynamic training, or a hybrid training.

11. The method of claim 6, wherein training the soon to expire analysis model comprises:
obtaining training item data;
generating a first soon to expire analysis model including a first processing pipeline wherein the machine learning model receives the training item data and wherein a heuristic model receives, as a first input, at least a portion of a first output from the machine learning model;
generating a second soon to expire analysis model including a second processing pipeline wherein the heuristic model receives the training item data and wherein the machine learning model receive, as a second input, at least a portion of a second output from the heuristic model;
measuring resource utilization for processing at least a portion of the training item data using the first soon to expire analysis model and second soon to expire analysis model; and
selecting one of the first soon to expire analysis model and second soon to expire analysis model as the soon to expire analysis model based on the resource utilization.

12. The method of claim 1, wherein the target data is one of: an expiration date for the item, a predetermined amount of time from a current date, or a scheduled inventory update date.

13. The method of claim 1, wherein a first instance of the item is available at a first location managed by the inventory controller and a second instance of the item is available a second location managed by the inventory controller, and
wherein the action to prevent the first instance of the item from remaining unused past the target date comprises configuring the inventory controller to, upon receiving a request to dispense the item, dispense the item from the first location.

14. A system, comprising:
at least one data processor; and
at least one memory storing instructions which, when executed by the at least one data processor, result in operations comprising:
receiving training data comprising historical data related to an item;
training a soon to expire analysis model using the training data to generate a trained soon to expire analysis model;
applying the trained soon to expire analysis model to determine the item stocked at one or more locations as being likely to remain unused past an expiration date; and
providing an output to perform an action to prevent the item from remaining unused past the expiration date.

15. The system of claim 14, wherein the action to prevent the item from remaining unused past the expiration date comprises moving, by an inventory controller, at least a portion of the item from the one or more locations to a use location and wherein the historical data comprises inventory changes from a plurality of locations.

16. The system of claim 14, wherein determining the item stocked at the one or more locations as being likely to remain unused past the expiration date comprises comparing a categorical value to a threshold, wherein the categorical value comprises one or more of: a unit cost, a usage velocity, and a moving speed of the item.

17. The system of claim 14, wherein the soon to expire analysis model comprises a machine learning model and one or more heuristic models, wherein the one or more heuristic models comprise a model defining an association between an item unit value and an item usage rate, an item value and the item usage rate, or an item unit value and a distance to earliest expiration date.

18. The system of claim 14, wherein training the soon to expire analysis model comprises:
determining whether the historical data satisfies a first threshold; and
in response to determining that the historical data satisfies the first threshold, executing the training using the machine learning model; or
in response to determining that the historical data fails to satisfy the first threshold, determining whether the historical data satisfies a second threshold; and
in response to determining that the historical data satisfies the second threshold, executing the training using one of the one or more heuristic models.

19. The system of claim 14, wherein training comprises any of a supervised training, an unsupervised training, a reinforced training, a dynamic training, or a hybrid training.

20. A non-transitory computer-readable medium storing instructions, which when executed by at least one data processor, result in operations comprising:
receiving training data comprising historical data related to an item;
training a soon to expire analysis model using the training data to generate a trained soon to expire analysis model;
applying the trained soon to expire analysis model to determine the item stocked at one or more locations as being likely to remain unused past an expiration date; and
providing an output to perform an action to prevent the item from remaining unused past the expiration date.
